(19) United States
(12) Patent Application Publication
(10) Pub. No.: US 2005/0239144 A1
(43) Pub. Date: Oct. 27, 2005

(54) Site-specific labelling of proteins using cyanine dye reporters

(76) Inventor: Graham Cotton, Lothian (GB)

Correspondence Address: AMERSHAM BIOSCIENCES;PATENT DEPARTMENT, 800 CENTENNIAL AVENUE, PISCATAWAY, NJ, 08855, US

(21) Appl. No.: 10/522,675

(22) PCT Filed: Jul. 28, 2003
(86) PCT No.: PCT/GB03/03196

§ 371 Date: Jan. 27, 2005

(30) Foreign Application Priority Data
Jul. 30, 2002 (GB) 0217556.0
Sep. 11, 2002 (US) 10241333

(51) Int. Cl. G01N 33/53

(52) U.S. Cl. 435/7.5; 530/409

(57) ABSTRACT

Disclosed are compounds of formula: 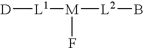
in which D is a dye selected from a cyanine dye or a derivative thereof; B is an affinity tag; F comprises a target bonding group selected from a carboxylic acid thioester group and a 1,2-aminothiol group; M is a group adapted for attaching to F; and L¹ and L² each independently comprise a group containing from 1 to 40 linked atoms selected from carbon atoms which may optionally include one or more groups selected from —NR′—, —O—, —CH═CH—, —CO—NH— and phenylenyl groups, where R′ is selected from hydrogen and C₁-C₄ alkyl. The invention also relates to methods that afford direct attachment of the cyanine dye reporter group to either the N-terminus or C-terminus of a synthetic or recombinant peptide or protein, and their derivatives, in a site-specific manner, coupled with purification of the resultant labelled molecule.

The present invention relates to reagents and methods for site-specific labelling of proteins using cyanine dyes as reporter molecules. In particular, the invention relates to new cyanine dye derivatives containing thioester activated groups and groups reactive with target molecules containing or derivatised to contain a thioester reactive moiety.

There is increasing interest in, and demand for, fluorescent reporters for use in the labelling and detection of biomolecules. Cyanine and related dyes such as rigidised cyanine dyes and squaraines offer a number of advantages over other fluorescent dye reagents and they are finding widespread use as fluorescent labels in such diverse areas as sequencing, microarrays, flow cytometry and proteomics. For example, U.S. Pat. No. 5,569,587 (Waggoner et al) discloses water soluble cyanine dye derivatives that possess reactive groups suitable for reaction with target molecules that contain, or are derivatised to contain, —OH, —NH₂, or —SH groups. The cyanine dyes are characterised by having very high extinction coefficients and favourable quantum yields. In addition, cyanine dyes possess good photostability and are not readily photobleached.

In many applications there is a need to form a permanent link, in the form of a covalent bond, between a fluorescent labelling dye and a target molecule such as a protein. The chemistry of peptide and protein labelling is well documented and a wide range of labelling reagents are now commercially available. For a review and examples of protein labelling using fluorescent labelling reagents, see “Non-Radioactive Labelling, a Practical Introduction”, Garman, A. J. Academic Press, 1997; “Handbook of Fluorescent Probes and Research Chemicals”, Haugland, R. P., Molecular Probes Inc., 1992).

Site-specific incorporation of a fluorescent label into a protein or peptide may be of considerable benefit in certain biochemical and biophysical studies, for example fluorescence resonance energy transfer, and protein structure and function studies. One method for the site-specific attachment of a fluorescent label into a target polypeptide utilises the native chemical ligation reaction. According to this procedure, an unprotected peptide fragment containing an N-terminal cysteine residue and a second unprotected peptide fragment containing an α-thioester group are chemoselectively ligated together at physiological pH, irrespective of their primary sequences, to generate an amide bond at the ligation site. For examples, see reviews by Cotton, G. J. and Muir T. W., Chem. Biol., (1999), 6, R247-260; Giriat, I., Muir, T. W. and Perler, F. B., Genetic Engineering, (2001), 23, 171-199; Muir, T. W., Syn. Lett., (2001), 6, 733-740.

Tolbert, T. J. and Wong, C-H. (Angew. Chem. Int. Ed., (2002), 41, 2171-2174) describe the preparation of fluorescein and biotin thioester derivatives and the reaction of these with N-terminal cysteine-containing recombinant proteins. Schuler, B. and Pannell, L. K. (Bioconjugate Chemistry, published on line, 18 Jul. 2002) reported the preparation of a benzyl thioester of Cy5M and subsequent reaction with a synthetic polypeptide containing an N-terminal cysteine residue.

However, there are no reports describing thioester derivatives of cyanine dyes in which the reporter is also linked covalently to an affinity tag. Use of such a reagent in reactions involving site specific labelling of proteins and peptides will be advantageous for subsequent separation and purification of the fluorescent dye-labelled target. The present invention therefore provides new cyanine dye reagents and methods that afford direct attachment of the cyanine dye reporter to either the N-terminus or C-terminus of a synthetic or recombinant peptide or protein and their derivatives, in a site-specific manner, coupled with purification of the resultant labelled molecule.

According to one aspect of the present invention, there is provided a compound comprising a cyanine dye or derivative thereof containing, at least one target bonding group selected from a carboxylic acid thioester group or a group suitable for covalent reaction with a thioester, characterised in that said compound includes an affinity tag covalently bound thereto.

Suitably, the compound is of formula (I): 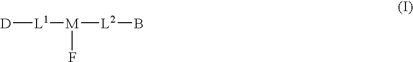
wherein:
D is a dye selected from a cyanine dye or a derivative thereof;
B is an affinity tag;
F comprises a target bonding group selected from a carboxylic acid thioester group and a 1,2-aminothiol group;
M is a group adapted for attaching to F; and
L¹ and L² each independently comprise a group containing from 1-40 linked atoms selected from carbon atoms which may optionally include one or more groups selected from —NR′—, —O—, —CH═CH—, —CO—NH— and phenylenyl groups, where R′ is selected from hydrogen and C₁-C₄ alkyl.

Suitably, there are 2 to 30 atoms in each of L¹ and L², preferably, 6 to 20 atoms.

Preferably, L¹ and L² are independently selected from the group:
{(CHR′)ₚ-Q-(CHR′)ᵣ}ₛ—
where Q is selected from: —CHR′—, —NR′—, —O—, —CH═CH—, —Ar— and —CO—NH—; R′ is hydrogen or C₁-C₄ alkyl, p is 0-5, r is 1-5 and s is 1 or 2.

Particularly preferred Q is selected from: —CHR′—, —O— and —CO—NH—, where R′ is hereinbefore defined.

In one embodiment L² is a cleavable linker and may additionally include group P which may be suitably selected from a chemically-cleavable group, an enzyme-cleavable group, or a photochemically-cleavable group. Suitable chemically cleavable groups include carbamate esters and carboxylate esters, which are both cleaved under basic conditions. Suitable enzyme cleavable groups may be selected from groups such as ester, amide and phosphodiester groups. Such groups are substrates for, and are hydrolysed by hydrolases, such as proteases, esterases and phospho-diesterases. Suitable photocleavable groups P for use in the compound of formula (I) may contain the 4,5-dialkoxy-2-nitrobenzyl alcohol linker (Holmes, C. P., and Jones, D. G., J. Org. Chem., (1995), 60, 2318-2319) or phenacyl linkers (Wang, S., J. Org. Chem., (1976), 41, 3258-3261). These groups undergo efficient photoreaction upon 300 nm illumination, resulting in the rapid cleavage of the dye molecule or dye-labelled protein from the affinity tag.

Suitably, the group M may be any suitable functional group adapted for attaching the target bonding group F. Preferably, M is selected from: 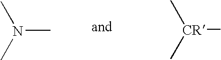
wherein R′ is hereinbefore defined.

Suitable affinity tags may be selected from biotin, desthiobiotin and metal chelating ligands such as his-tag and iminodiacetic acid, nitrilotriacetic acid and the like. Preferred affinity tags are selected from biotin and desthiobiotin.

In one embodiment of the present invention, the target bonding group F is a carboxylic acid thioester of formula: 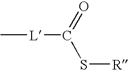
wherein L′ is a bond or is a group containing from 1-30 linked atoms selected from carbon atoms and optionally one or more groups selected from —NH—, —O— and —CO—NH—; and R″ is C₁-C₄ alkyl, C₆-C₁₀ aryl, or C₇-C₁₅ aralkyl, which may be optionally substituted with sulphonate; or is the group —(CH₂)₂CONH₂. In the case where L′ is a bond, the target bonding group F is attached directly to group M.

In an alternative embodiment, the target bonding group F is a 1,2-aminothiol group of formula: 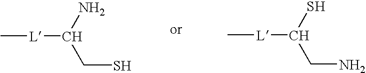
wherein L′ is hereinbefore defined.

Thus, the present invention provides fluorescent labelling reagents comprising a cyanine dye or derivative thereof, that are modified by incorporating a target bonding group and an affinity tag into the molecule. The target bonding group may be selected from a carboxylic acid thioester group or a 1,2-aminothiol group. Where the target bonding group is a thioester group, it is selectively reactive with a 1,2-aminothiol group on a target molecule, suitably a protein or peptide, or a derivative thereof. In the alternative, the cyanine dye may contain a 1,2-aminothiol group for reaction with a thioester group on the target. The incorporation of a reactive thioester or, alternatively, a 1,2-aminothiol functionality into the chemical structure of the reporter molecule enables the target molecule to be directly labelled in a convenient one step process. According to the methods of the invention, labelling of peptides and proteins is site-specific, irrespective of the composition of the primary sequence. By generating the target primary sequence with either an N-terminal cysteine or a thioester functionality, site-specific labelling can be achieved directly, by incubating the target with the appropriate derivative of the cyanine dye, suitably, the thioester and 1,2-aminothiol derivatives respectively. Furthermore, inclusion of an affinity tag in the labelling reagent allows subsequent purification of the fluorescent dye labelled protein or peptide.

Suitably, the cyanine dye or cyanine dye derivative may be selected from cyanine dyes, rigidised cyanine dyes and squaraine dyes, provided that the dye incorporates at least one carboxylic acid thioester group, or a group suitable for covalent reaction with a thioester. Table 1 shows some examples of cyanine dyes, having particular excitation (Abs) and emission (Em) characteristics.

TABLE 1

| Dye | Fluorescence Colour | Abs (nm) | Em (nm) |
|---|---|---|---|
| Cy2 | Green | 489 | 506 |
| Cy3 | Orange | 550 | 570 |
| Cy3.5 | Scarlet | 581 | 596 |
| Cy5 | Far red | 649 | 670 |
| Cy5.5 | Near-IR | 675 | 694 |
| Cy7 | Near-IR | 743 | 767 |

In one embodiment according to the first aspect, the compound has the formula (II): 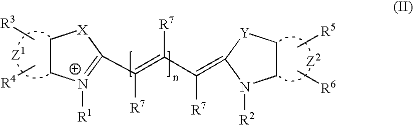
wherein:
groups R³ and R⁴ are attached to the Z′ ring structure and groups R⁵ and R⁶ are attached to the Z² ring structure;
n is an integer from 1 to 3;
Z¹ and Z² independently represent the atoms necessary to complete one ring or two fused ring aromabc or heteroaromatic systems, each ring having five or six atoms selected from carbon atoms and optionally no more than two atoms selected from oxygen, nitrogen and sulphur;
X and Y are the same or different and are selected from: >CR⁸R⁹, oxygen, sulphur, —CH═CH—, >N—W wherein N is nitrogen and W is selected from hydrogen and the group R¹⁰;
at least one of groups R¹, R², R³, R⁴, R⁵, R⁶, R⁸, R⁹ and R¹⁰ is the group: 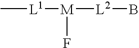
   where B, F, M, L¹ and L² are hereinbefore defined; groups R⁷ are independently selected from hydrogen and C₁-C₄ alkyl which may be unsubstituted or substituted with aryl, or two or more of R⁷ together with the group: 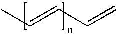
   form a hydrocarbon ring system substituted with R⁷ and which may optionally contain a heteroatom selected from —O—, —S— or >NR⁷, wherein R⁷ and n are hereinbefore defined;
remaining groups R³, R⁴, R⁵ and R⁶ are independently selected from the group consisting of hydrogen, halogen, amide, cyano, nitro, mono- or di-C₁-C₆ alkyl-substituted amino, carbonyl, carboxyl, C₁-C₆ alkyl, C₁-C₆ alkoxy, aryl, heteroaryl, aralkyl and the group —(CH₂)ₘ—Y where Y is selected from sulphonate, sulphate, phosphonate, phosphate and quaternary ammonium and m is zero or an integer from 1 to 6;
remaining groups R⁸, R⁹ and R¹⁰ are independently C₁-C₆ alkyl; and remaining groups R¹ and R² are independently selected from hydrogen, C₁-C₁₀ alkyl, the group —(CH₂)ₘ—Y wherein Y and m are hereinbefore defined, and benzyl which may be unsubstituted or substituted by up to two nitro groups.

In a second embodiment according to the first aspect, the compound has the formula (III): 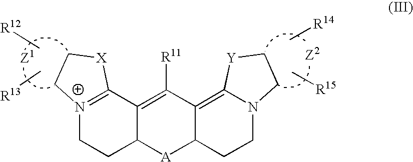
wherein
groups R¹², R¹³, R¹⁴ and R¹⁵ are attached to the rings containing X and Y or, optionally are attached to atoms of the Z¹ and Z² ring structures;
Z¹ and Z² independently represent the atoms necessary to complete one ring or two fused ring aromatic or heteroaromatic systems, each ring having five or six atoms selected from carbon atoms and optionally no more than two atoms selected from oxygen, nitrogen and sulphur;
X and Y are the same or different and are selected from: >CR⁸R⁹, oxygen, sulphur, —CH═CH—, >N—W wherein N is nitrogen and W is selected from hydrogen and the group R¹⁰;
A is selected from O and NR¹⁶ where R¹⁶ is the substituted amino radical: 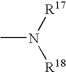
   at least one of groups R⁸, R⁹, R¹⁰ R¹¹, R¹², R¹³, R¹⁴, R¹⁵, R¹⁷ and R¹⁸ is the group: 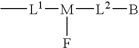
   where B, F. M, L¹ and L² are hereinbefore defined;
remaining groups R¹¹, R¹², R¹³, R¹⁴ and R¹⁵ are independently selected from the group consisting of hydrogen, halogen, amide, cyano, nitro, amino, mono- or di-C₁-C₆ alkyl-substituted amino, carbonyl, carboxyl, C₁-C₆ alkyl, C₁-C₆ alkoxy, aryl, heteroaryl, aralkyl and the group —(CH₂)ₘ—Y where Y is selected from sulphonate, sulphate, phosphonate, phosphate and quaternary ammonium and m is zero or an integer from 1 to 6;
remaining groups R⁸, R⁹ and R¹⁰ are independently C₁-C₆ alkyl; remaining group R¹⁷ is selected from hydrogen, C₁-C₄ alkyl and aryl; and remaining group R¹⁸ is selected from C₁-C₆ alkyl, aryl, heteroaryl, an acyl radical having from 2-7 carbon atoms, and a thiocarbamoyl radical.

Suitably, in the compounds according to formula (II) and (III), Z¹ and Z² may be selected independently from the group consisting of phenyl, pyridinyl, naphthyl, anthranyl, indenyl, fluorenyl, quinolinyl, indolyl, benzothiophenyl, benzofuranyl and benzimidazolyl moieties. Additional one, or two fused ring systems will be readily apparent to the skilled person. Preferably, Z¹ and Z² are selected from the group consisting of phenyl, pyridinyl, naphthyl, quinolinyl and indolyl moieties. Particularly preferred Z¹ and Z² are phenyl and naphthyl moieties.

Suitably, at least one of the groups R of the compounds of formula (II) and (III) is a water solubilising group for conferring a hydrophilic characteristic to the compound. Solubilising groups, for example, sulphonate, sulphonic acid and quaternary ammonium, may be attached directly to the aromatic ring structures Z¹ and/or Z² of the compounds of formula (II) and (III). Alternatively, solubilising groups may be attached by means of a C₁ to C₆ alkyl linker chain to said aromatic ring structures and may be selected from the group —(CH₂)ₘ—Y where Y is selected from sulphonate, sulphate, phosphonate, phosphate, quaternary ammonium and carboxyl; and m is hereinbefore defined. Alternative solubilising groups may be carbohydrate residues, for example, monosaccharides, or polyethylene glycol derivatives. Examples of water solubilising constituents include C₁-C₆ alkyl sulphonates, such as —(CH₂)₃—SO₃— and CH₂)₄—SO₃—. However, one or more sulphonate or sulphonic acid groups attached directly to the aromatic ring structures of a dye of formula (II) or (III) are particularly preferred. Water solubility may be advantageous when labelling proteins.

In one embodiment the compound of formula (I) is a fluorescent reporter molecule. In this embodiment, none of the substituent groups R in the compounds of formula (II) and (III) contains a nitro group.

In another embodiment, the compound of formula (I) is non-fluorescent or substantially non-fluorescent dye wherein at least one of the groups R attached to the aromatic ring structures of the compounds of formula (II) and (III) comprises at least one nitro group. In this embodiment, suitably, the at least one nitro group may be attached directly to the Z¹ and/or Z² ring structures. In the alternative, a mono- or di-nitro-substituted benzyl group may be attached to the Z¹ and/or Z² ring structures which optionally may be further substituted with one or more nitro groups. The non-fluorescent or substantially non-fluorescent cyanine dye or cyanine dye derivatives according to the invention may be used to label one component of a fluorescent donor/acceptor pair in assays involving the detection of binding and/or cleavage events in reactions involving biological molecules, as described in EP 1086179 B1 (Amersham Biosciences UK Limited).

In the embodiments according to the first aspect:
i) Aryl is an aromatic substituent containing one or two fused aromatic rings containing 6 to 10 carbon atoms, for example phenyl or naphthyl, the aryl being optionally and independently substituted by one or more substituents, for example halogen, straight or branched chain alkyl groups containing 1 to 10 carbon atoms, aralkyl and alkoxy for example methoxy, ethoxy, propoxy and n-butoxy;
ii) Heteroaryl is a mono- or bicyclic 5 to 10 membered aromatic ring system containing at least one and no more than 3 heteroatoms which may be selected from N, O, and S and is optionally and independently substituted by one or more substituents, for example halogen, straight or branched chain alkyl groups containing 1 to 10 carbon atoms, aralkyl and alkoxy for example methoxy, ethoxy, propoxy and n-butoxy;
iii) Aralkyl is a C₁-C₆ alkyl group substituted by an aryl or heteroaryl group;
   iv) Halogen and halo groups are selected from fluorine, chlorine, bromine and iodine.

By virtue of the target bonding group F, the compounds according to the present invention are useful for covalently labelling target biological materials in a site specific manner for applications in biological detection systems. Suitable target materials include proteins, post-translationally modified proteins, peptides, antibodies, antigens, and protein-nucleic acids (PNAs). The reporter moiety may also be conjugated to species which can direct the path of the reporter within or aid entry to or exit from cells (live or dead); such as for example, long alkyl residues to allow permeation of lipophilic membranes, or intercalating species to localise a reporter in a nucleus or other cellular enclave containing double-stranded DNA.

In a second aspect, there is provided a method for labelling a protein of interest wherein said protein contains or is derivatised to contain an N-terminal cysteine, the method comprising:
i) adding to a liquid containing said protein a compound of formula (I): 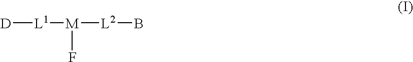
   wherein:
D is a dye selected from a cyanine dye or a derivative thereof;
B is an affinity tag;
F comprises a target bonding group selected from a carboxylic acid thioester group and a 1,2-aminothiol group;
M is a group adapted for attaching to F; and
L¹ and L² each independently comprise a group containing from 1-40 linked atoms selected from carbon atoms which may optionally include one or more groups selected from —NR′—, —O—, —CH═CH—, —CO—NH— and phenylenyl groups, where R′ is selected from hydrogen and C₁-C₄ alkyl; and
ii) incubating said compound with said protein under conditions suitable for labelling said protein.

Suitably, there are 2 to 30 atoms in each of L: and L², preferably, 6 to 20 atoms.

Preferably, L¹ and L² are independently selected from the group:
—{(CHR′)ₚ-Q-(CHR′)ᵣ}ₛ—
where Q is selected from: —CHR′—, —NR′—, —O—, —CH═CH—, —Ar— and —CO—NH—; R′ is hydrogen or C₁-C₄ alkyl, p is 0-5, r is 1-5 and s is 1 or 2.

Particularly preferred Q is selected from: —CHR′—, —O— and —CO—NH—, where R′ is hereinbefore defined.

Preferred compounds of formula (I) for use in labelling a target protein are those having formula (II) or (III) as hereinbefore defined.

Covalent labelling using compounds of the present invention may be accomplished with a target having at least one carboxylic acid thioester group or 1,2-aminothiol group as hereinbefore defined. The target may be incubated with an amount of a compound of the present invention having at least one group F as hereinbefore defined that can covalently bind with the complementary group of the target material. The target material and the compound of the present invention are incubated under conditions and for a period of time sufficient to permit the target material to covalently bond to the compound of the present invention. Thus, for example, the thioester group F may be reacted and form a covalent bond with any of the above target materials that contains, or has been derivatised to contain, a 1,2-amino thiol group. These methods and the products resulting from them, for example, reporter-labelled biomolecules are envisaged as further aspects of the invention.

Suitably, the protein of interest may be selected from the group consisting of antibody, antigen, protein, peptide, microbial materials, cells and cell membranes.

In a particular embodiment according to the second aspect, there is provided a method of separating and/or purifying the dye-labelled protein of interest by affinity chromatography utilising the affinity of the affinity tag moiety for an immobilised ligand (or specific binding partner) attached to a support material. Affinity chromatography provides a quick and convenient method to enable the separation of labelled and unlabelled protein molecules under physiological conditions. Proteins labelled with an affinity tag can be selectively bound to an affinity column and any unreacted protein removed by washing the column. Suitable specific binding moieties include avidin or streptavidin (for a biotin tag); immobilised metal ions, for example, Cu(II), Ni(II), Fe(II) and Fe(III) (for His-tag or iminodiacetic acid). Methods for affinity purification of proteins will be well known to the skilled person, see for example Ostrove, S, Methods in Enzymology, (1990), Vol 182, page 357.

In a typical labelling procedure, a target peptide or protein containing an N-terminal cysteine residue is agitated with an excess of a cyanine dye thioester derivative, e.g. Cy5-MESNA (Cy5-mercaptoethanesulphonic acid ester), in phosphate buffer (typically 200 mM NaCl, 200 mM sodium phosphate) at ˜pH 7.3-7.4 containing ˜1.5% MESNA. The concentration of the target polypeptide in the labelling reaction is generally between 100 μM to 10 mM, whilst the Cy5-MESNA is generally present in excess, for example 1.5 to 3-fold molar excess. When the target polypeptide concentration is relatively low the concentration of Cy5-MESNA is usually maintained at or above 1 mM. Generally, for labelling small peptides a solution of Cy5-MESNA and MESNA cofactor is directly added to the lyophilised target.

Typically, for site specific labelling of proteins and large polypeptides using the reagents of the present invention, the target is first exchanged into an appropriate buffer, which is known not to affect the labelling reaction. An equal volume of a solution of Cy5-MESNA and MESNA thiol cofactor in ligation buffer is then added to the protein to give the desired final concentration of the reactants. The reaction mixture is agitated overnight at room temperature. The reaction time may be lowered to less than one hour for high reactant concentrations or, if the stability of the target polypeptide is an issue, the labelling reaction may be performed efficiently at 4° C. On completion of the labelling reaction, dithiothreitol (DTT) is added to a final concentration of −50 mM and the desired material isolated by affinity chromatography.

Various different denaturants, organic solvents and detergents may be added to the reaction buffer when performing native chemical ligation and expressed protein ligation reactions, to aid the ligation of the peptide fragments and/or stabilise the reactants or products. Such reagents may be utilised in the labelling reaction to increase product yield if necessary. Examples include, but are not limited to guanidinium chloride, urea, dimethylformamide, dimethylsulphoxide, acetonitrile, triton X-100, octyl glucoside, 1,6-hexanediol and glycerol.

The ligation reaction using the derivatised cyanine dye according to the present invention may be optimally performed at between pH 7.0 and pH 8.0 and at temperatures varying between 4° C. and 37° C. It is envisaged that such a range of conditions are compatible to the site-specific labelling reaction described herein.

The advantage of the present method is that it enables the introduction of an extrinsic label into a proteinacious substrate in a regioselective and specific manner, thus minimising any detrimental effects that labelling may have on the biological function of the protein. The importance of controlling stoichiometry of labelling is important where dye overload may interfere with biological activity. In addition, if this controlled labelling stoichiometry is directed towards a single terminal site, rather than towards an internal site, this may have the benefit of further maintaining the biological viability of the labelled species.

The invention is further illustrated by reference to the following examples and figure in which:

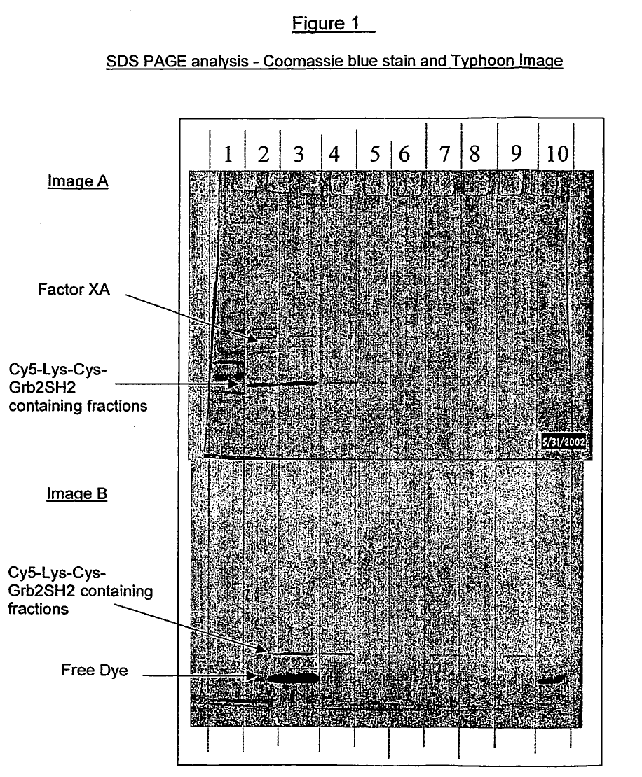

FIG. 1 illustrates the products from the labelling reaction of an N-terminal cysteine derivative of the Grb2SH2 domain with the thioester derivative, α-D-desthiobiotin-ε-Cy5-L-lysine-MESNA according to Examples 3 and 4.

### EXPERIMENTAL

1. 2-[(1E,3E,5E)-5-(3,3-Dimethyl-1-{6-oxo-6-[(2-sulphoethyl)thio]hexyl}-5-sulfo-1,3-dihydro-2H-indol-2-ylidene)penta-1,3-dienyl]-1-ethyl-3.3-dimethyl-5-sulfo-3H-indolium

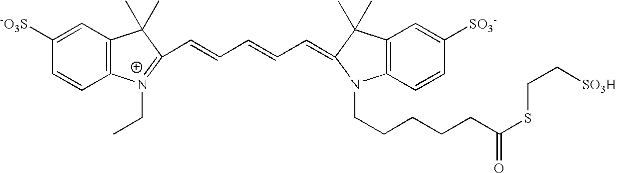

To Cy™5 mono acid (47 mg, 0.062 mmol) in a solution of 7-azobenzotriazolyoxytris(pyrrolidino)phosphonium hexafluorophosphate (PyAOP, 66 mg, 0.127 mmol) in anhydrous dimethylformamide (DMF, 1 ml) was added anhydrous di-isopropylethylamine (DIEA)(30 μl, 0.1724 mmol) and mixed for 5 minutes. The activated dye solution was then added to a stirred solution of 2-mercaptoethanesulphonic acid, sodium salt (MESNA, 40 mg, 0.243 mmol) in DMF (2 mls) and DIEA (30 μl, 0.1724 mmol) under a dry nitrogen atmosphere. To this mixture was added as a solid, dried 4A molecular sieves (˜1 g, <5 micron, activated powder). The mixture was stirred under a dry nitrogen atmosphere, at room temperature, in the dark overnight. Thin layer chromatography analysis (reverse phase C18 plates, eluents water/acetonitrile (70:30, containing 0.1% TFA) indicated a major component, Rfₜₕᵢₒₑₛₜₑᵣ=0.25) with no trace of starting material (Rf_{acid}=0.12).

The molecular sieves were removed by filtration and filtrate was added dropwise into an excess of ethyl acetate, the blue solid was filtered off and was purified by reverse phase-high performance liquid chromatography (RP-HPLC); [Phenomenex Prodigy C18 column; 15% B-30% B over 30 mins @ 20 ml/min; eluent A=0.1% TFA/water, eluent B=0.1% TFA.MeCN, UV detection at 650 nm]. The product was isolated as a dark blue/purple solid (40 mg, 0.0513 mmol, 83% yield).

Accurate mono-isotopic mass: C₃₅H₄₅O₁₀N₂S₄ requires 781. Found Maldi Tof, LC-MS found mass: M+781.25. δ H (300 MHz, d6-DMSO): 8.37 (t, 1H), 8.36 (t, 1H), 7.83 (d, 1H), 7.82 (d, 1H), 7.67(dd, 1H), 7.64 (dd, 1H), 7.36 (d, 1H), 7.33 (d, 1H), 6.61 (t, 1H), 6.38 (d, 1H), 6.28 (d, 1H), 4.15 (m, 2H), 4.08 (t, 2H), 3.06 (m, 2H), 2.63 (m, 2H), 2.56 (t, 2H), 1.64 (m, 2H), 1.28(t, 3H, 7.1), 1.40 (m, 2H). λₘₐₓ (abs>=647 nm. (ε(H₂O)=230,000 M⁻¹ cm⁻¹).

2. Determination of Specificity of Labelling using 2-[(1E,3E,5E)-5-(3,3-dimethyl-1-{6-oxo-6-[(2-sulphoethyl)thio]hexyl}-5-sulfo-1,3-dihydro-2H-indol-2-ylidene)penta-1,3-dienyl]-1-ethyl-3,3-dimethyl-5-sulfo-3H-indolium

2.1 Preparation of Cv5-Cys-Gly-Leu-Asp-Lys-Arg-Gly-Cys-Gly-NH₂

i) Synthesis of H-Cys(Trt)-Gly-Leu-Asp(OtBu)-Lys(Boc)Arg(Pmc)-Gly-Cys(Trt)-Gly-rink amide resin

H-Cys(Trt)-Gly-Leu-Asp(OtBu)Lys(Boc)-Arg(PmcyGly-Cys(Trt)-Gly-rink amide resin was synthesised using a commercially available Applied Biosystems Model 433A automated peptide synthesiser using FastMoc™ chemistry, following the instrument manufacturer's recommended procedures throughout. The peptide was synthesised on a 0.25 millimolar scale employing O-(benzotriazol-1-yl)-1,1,3,3-tetramethyluronium hexafluorophosphate (HBTU) as the activating agent.
ii) H-Cys-Gly-Leu-Asp-Lys-Arg-Gly-Cys-Gly-NH₂

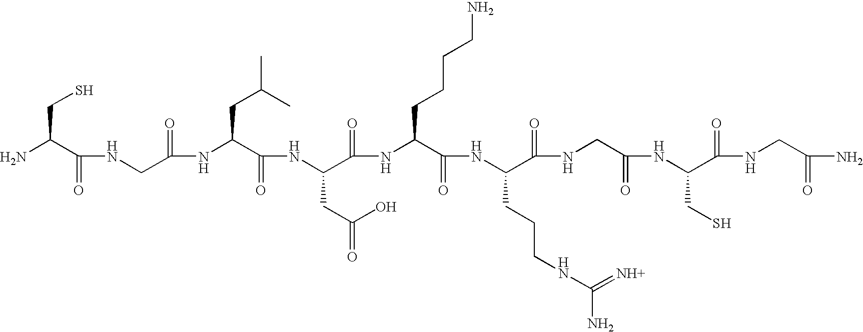

H-Cys(Trt)-Gly-Leu-Asp(OtBu)-Lys(Boc)-Arg(Pmc)-Gly-Cys(Trt)Gly-rink amide resin (100 mg, theoretical loading 0.36 mmol/g) was deprotected and cleaved from solid phase in 95% trifluoroacebc acid (TFA)/2.5% triisopropylsilane (TIS)/2.5% water (3 mis) at room temperature for 2 hours. The crude product was precipitated into a 10 fold excess of cold diethyl ether, centrifuged at 2500 rpm for 5 minutes and the ether decanted off. The crude peptide was washed twice more with ether and was purified by reverse phase-high performance liquid chromatography (RP-HPLC) [Phenomenex Jupiter C18 column, eluent A: 0.1% TFA/water, eluent B: 0.1% TFA/acetonitrile, gradient: 0-73% B over 30 mins @1 ml/min, detection at 214 nm]. The product was isolated and lyophilised to afford a colourless fluffy solid (21 mg by weight, 60%). Mono-isotopic mass: 906.4. Found mass (LC-MS): MH+@ 907.3;
M+Na @ 929.6; >95% pure as judged by RP-HPLC @ 214 nm (Phenomenex Jupiter C18 column, eluent A: 0.1% TFA/water, eluent B: 0.1% TFA/acetontrile, 5-50% B over 25 mins @ 1 ml Imin, UV detection at 650 nm).
   iii) Cv5-Cys-Gly-Leu-Asp-Lvs-Arg-Gly-Cys-Gly-NH₂

To solid H-Cys-Gly-Leu-Asp-Lys-Arg-Gly-Cys-Gly-NH2 (3.0 mg by weight, 0.0033 mmol) was added a solution of Cy5-MESNA (3.5 mg, 0.0045 mmol) in 200 mM phosphate buffer, 200 mM NaCl pH 7.2 containing 1.5% 2-mercaptoethanesulphonic acid, sodium salt (400 μl). The reaction mixture was stirred on rollers for 30 minutes at room temperature in darkness. During incubation, a blue precipitate formed, which re-dissolved on addition of acetonitrile (40 μl).

500 mM DTT (200 μl) in 200 mM phosphate buffer; 200 mM NaCl pH 7.2 (0.5 mis, 0.0025 mmol) was then added to the reaction mixture, with complete mixing and was stirred for a further 30 minutes at room temperature in the dark. The crude reaction mixture was then purified by RP-HPLC [Phenomenex Jupiter C18 column, eluent A: 0.1% TFA/water, eluent B: 0.1% TFA/acetontrile, gradient; 20-35% B over 30 mins at 4 ml/min, detection at 650 nm and 214 nm]. The product was isolated and lyophilised as a blue fluffy solid (1.6 mg by UVNIS at 650 nm; 50% Yield; 98% pure as judged by RP-HPLC at 650 nm. Mono-isotopic mass C₆₇H₁₀₁N₁₆O₁₈S₄ requires 1545.636. Found (LC-MS) M+1545.7.

2.2 Characterisation of Labelled Peptide

i) Ellman's Test on Cy5-Cys-Gly-Leu-Asp-Lys-Arg-Gly-Cys-Gly-NH₂

A sample of Cy5-Cys-Gly-Leu-Asp-Lys-Arg-Gly-Cys-Gly-NH₂ was dissolved in 100 mM sodium phosphate buffer; 1 mM EDTA pH 7.27 (stock buffer) to afford a 0.3 μM peptide stock by UVNIS at 650 nm.

0.3 μM peptide stock (40 μl) and 10 mM 5,5′-dithiobis(2-nitrobenzoic acid (DTNB) in 100 mM sodium phosphate buffer; 1 mM EDTA pH 7.27 (50 μl) were mixed together in stock buffer (910 μl) to afford a green solution. The absorbance at 412 nm (due to generation of TNB²⁻) was recorded against a DTNB blank [10 mM DTNB stock (50 μl) in stock buffer (950 μl]. Using the known molar absorption coefficient of TNB²⁻ (14150M⁻¹ cm⁻¹), the thiol concentration was determined as 655 μM, approximately twice the peptide concentration, confirming two free thiol groups. [SH]=[A412 nm (sample)-A412 nm(reference)/ε (TNB²⁻)

ii) Enzyme Digestion of Cy5-Cys-Gly-Leu-Asp-Lys-Arg-Gly-Cys-Gly-NH₂

To a solution of Cy5-Cys-Gly-Leu-Asp-Lys-Arg-Gly-Cys-Gly-NH₂ (180 μg by UV/VIS at 650 nm) in TRIS buffer pH 8.0 (100 μl) containing 10% acetonitrile was added Asp-N (2 μg) in TRIS buffer pH 8.0 (70 μl). The reaction mixture was stirred at room temperature in the dark for 4 hours. The reaction mixture was treated with 250 mM Tris (2-carboxyethyl)phosphine, HCL (TCEP) in TRIS buffer pH 8.0 (55 μl) for 30 minutes. The reduced reaction mixture was then diluted 1:5 with 0.1% TFA in water and purified by reverse phase HPLC [Phenomenex Jupiter C18, eluent A: 0.1% TFA/water, eluent B: 0.1% TFA/acetonitrile, 5-50% B over 30 mins @ 1 ml/min, UV at 214 nm, 650 nm]. The two components of the reaction mixture were identified as: Cy5-Cys-Gly-Leu-OH, mono-isotopic mass: C₄₄H₆₀N₅O₁₁S₃ requires 930.3451. Found mass (MALDI Tof): M+930.0 and H-Asp-Lys-Arg-Gly-Cys-Gly-NH₂, monoisotopic mass: C₂₃H₄₃N₁₁O₈S requires 633.3016, found mass (MALDI-Tof): M+633.0.
3. Preparation of α-D-Desthiobiotin-ε-Cy5-L-lysine-MESNA [N⁶-(6-{(2Z)-2-[(2E,4E)-5-(1-ethyl-3,3-dimethyl-5-sulfo-3H-indolium-2-yl)penta-2,4-dienylidenel-3.3-dimethyl-5-sulfo-2,3-dihydro-1H-indol-1-yl}hexanoyl)-N²-(6-{(6-(5-methyl-2-oxoimidazolidin-4-yl)hexanoyl]amino}hexanoyl)lysylthio ethane-2-sulfonic acid] 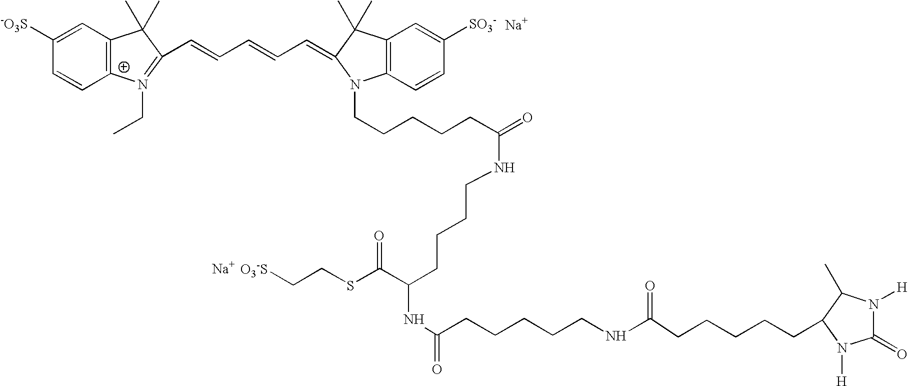
3.1 Preparation of α-Fmoc-c-Cy5-L-lysine-OH [2-[(1E,3E)-5-(1-(6-[(5-carboxy-5-{[(9H-fluoren-9-ylmethoxy)carbonyl]amino}pentyl)amino]-6-oxohexy}-3,3-dimethyl-5-sulfo-1,3-dihydro-2H-indol-2-ylidene)-1,3-pentadienyl]-1-ethyl-3,3-dimethyl-5-sulfo-3H-indolium salt]

Cy5 mono free acid potassium salt (Amersham Pharmacia Biotech Ltd) (450 mg, 0.65 mmol) and DIEA (720 μl) were dissolved in anhydrous dimethylsulphoxide (18 ml). To this was added O-(N-succinimidyl)-N,N,N′,N′-bis(tetramethylene)-uronium hexafluorophosphate (666 mg, 1.6 mmol) and the reaction mixture stirred at room temperature for 1 hr after which time negligible starting material remained by TLC (RPC₁₈, 1:1 methanol:water). The reaction mixture was slowly poured into diethyl ether to precipitate the product; Cy5 mono NHS ester, which was filtered off, washed with ethyl acetate and dried in vacuo. The product was re-dissolved in anhydrous dimethylsulphoxide (18 ml) and DIEA (720 μl) added. Fmoc-lysine-OH (360 mg, 0.98 mmol) was suspended in a mixture of phosphate buffer (pH 7.4) (9 ml) and dimethylsulphoxide (9 ml). The suspension was slowly added to the solution of Cy5 NHS ester. The reaction mixture was stirred at room temperature for 12 hours. TLC (RPC₁₈, 2:3 methanol:water) showed the disappearance of starting material and the formation of a new product spot.

The product was purified by HPLC (Dynamax C18 column (50×4.14 cm); flow rate 25 ml/min; gradient of 20 to 80% B over 80 mins (eluent A=0.1% TFA in water and eluent B=0.1% TFA in acetonitrile); detection at 650 nm. The fractions containing the desired product were pooled and most of the solvent removed under reduced pressure, the residue was freeze dried. The product; α-Fmoc-c-Cy5-L-lysine-OH was obtained as a fluffy cyan solid (487 mg, 74%). MS (MALDI TOF) found 1008(M⁺); [theoretical (C₅₄H₆₄N₄O₁₁S₂) 1009]. ¹H NMR (200 MHz D₆DMSO) 1.27(t, 3H, CH₃), 1.35 (m, 4H, CH₂, CH₂), 1.55 (m's, 4H, CH₂, CH₂), 1.7 (s, 12H, (CH₃)₂), 1.78 (m, 2H, CH₂), 2.05 (t, 2H, CH₂), 3.0, (m, 2H, CH₂), 3.92 (m, 1H CH amino acid), 4.11 (m, 4H, N—CH₂, N⁺CH₂), 4.27 (m 3H O—CH₂, CH fluorenyl), 6.3 (d, 2H, α, α′ methine), 6.59, (t, 1H, γ methine) 7.28-7.48 (m's, 6H, Fmoc and indole Ar), 7.65 (d, 2H, fluorenyl Ar), 7.73 (d, 2H, fluorenyl Ar), 7.85 (s, 2H, indole Ar), 7.9 (d, 2H, indole Ar), 8.38 (t, 2H, β,β′ methine).

3.2 Preparation of E-Cy5-L-lysine-OH [N⁶-(6-{(2E)-2-[(2E,4E)-5-(1-ethyl-3,3-dimethyl-5-sulfo-3H-indolium-2-yl)penta-2,4-dienylidene]-3,3-dimethyl-5-sulfo-2,3-dihydro-1H-indol-1-yl}hexanoyl)lysine]

α-Fmoc-c-Cy5-L-lysine-OH (100 mg, 0.1 mmol) was deprotected in a mixture of 20% piperidine in NMP (2 ml). TLC (RP C18; 1:1 MeOH:water) showed the formation of a new product spot, rf=0.92 as compared to that of the starting material, rf=0.46. The piperidine was removed under reduced pressure and the dye precipitated by pouring the reaction mixture into diethyl ether. The product was filtered off and washed with dichloromethane and then ethyl acetate to remove the yellow Fmoc derived by-product. The product was dissolved in water, filtered and then purified by HPLC; [Vydac Protein and Peptide C18 column; 0-50% B over 45 mins at 10 ml/min; eluent A=0.1% TFA/water, eluent B=0.1% TFA/MeCN, detection at 215 nm). Fractions containing the desired product were combined and the solvents removed under reduced pressure to leave a blue residue. The residue was triturated with ethyl acetate and the resultant solid dried under vacuum at 40° C. The product; c-Cy5-L-lysine-OH was obtained as a dark blue solid (43 mg, 48%). Analytical HPLC AKTA analysis; Phenomenex C18 column; 0-50% B over 30 mins at mli/min; eluent A=0.1% TFA/water, eluent B=0.1% TFA/MeCN, detection at 650 nm; rt=20.22 mins. MS (MALDI TOF) found 785 (M⁺); [theoretical (C₃₉H₅₃N₄O₉S₂) 785]. ¹H NMR (300 MHz D₆DMSO) 1.26 (t, 3H, CH₃), 1.52 (m, 4H, CH₂, CH₂), 1.62 (m, 4H, CH₂, CH₂), 1.69 (S, 12H, (CH₃)₂), 2.02 (m, 2H, CH₂), 2.92 (m, 2H, CH₂), 3.85 (m, 1H, CH amino acid), 4.10 (m, NCH₂, N⁺CH₂), 6.29 (d, 1H a methine), 6.34 (d, 1H, α′ methine), 6.58 (t, 1H, γ methine), 7.32 (m, 2H, indole Ar), 7.82 (d, 2H, indole Ar), 8.04 (m, 3H, NH₃⁺), 8.37 (t, 2H, β,β′ methine).

3.3 Preparation of D-Desthiobiotinamidocaproic Acid

D-Desthiobiotin (250 mg, 1.17 mmol) was dissoved in anhydrous dimethylsulphoxide (2 ml). To this solution was added PyAOP (610 mg, 1.17 mmol) and DIEA (200 μl, 1.15 mmol). The mixture was stirred under nitrogen at RT for 3 hrs before adding 6-aminocaproic acid (153 mg, 1.17 mmol) and a further amount of DIEA (200 μl, 1.15 mmol). The reaction mixture was stirred for a further 4 hrs. TLC (RP C18; 1:2 MeOH:water; detection by cinnamaldehyde staining) showed the formation of a new product spot, rf=0.63 as compared to the starting material, rf=0.76). The reaction mixture was poured into excess diethyl ether to give a brown oil. The oil was triturated with ethyl acetate until an off-white solid was obtained. The product was filtered off and purified by HPLC [Vydac Protein and Peptide C18 column; 0-50% B over 30 mins at 10 ml/min; eluent A=0.1% TFA/water, eluent B=0.1% TFA/MeCN, detection at 215 nm). Fractions containing the desired product were pooled and the solvents removed under reduced pressure. The residue was triturated with ethyl acetate to give a white solid. The product was filtered off and dried under reduced pressure at 40° C. The product; D-desthiobiotinamidocaproic acid, was obtained as a white solid (48 mg, 12.5%). MS (MALDI TOF) found 327 (M+); [theoretical (C₁₆H₂₉N₃O₄) 327]. ¹H NMR (300 MHz D₆DMSO) 0.96 (d, 3H, CH₃), 1.25 (m, 6H, CH₂, CH₂, CH₂), 1.34 (m, 4H, CH₂, CH₂), 1.48 (m, 4H, CH₂, CH₂), 2.03 (m, 2H, C(O)CH₂), 2.60 (m, 2H, C(O)CH₂), 3.01 (m, 2H, NHCH₂), 3.47 (m, 1H, CH), 3.60 (m, 1H, CH), 6.11 (s, 1H, NH), 6.29 (s, 1H, NH), 7.71 (s, 1H, NH).

3.4 Preparation of D-Desthiobiotinamidocaproate N-hydroxy Succinimidyl Ester

D-Desthiobiotinamidocaproic acid (48 mg, 0.147 mmol) was dissolved in DMF (1 ml) and N,N,N′,N′-bis(tetramethylene)-O-(N-succinimidyl)uronium hexafluorophosphate (HSPyU) (90 mg, 0.21 mmol) and DIEA (40 μl, 0.23 mmol) were added. The reaction mixture was stirred under nitrogen at RT for 6 hrs, TLC (RP C18; 1:2 MeOH:water; materials detected by cinnamaldehyde staining) showed the formation of a new product at the base line as compared to the starting material, rf=0.68. The reaction mixture was poured into diethyl ether to give a brown gum. The supernatant was decanted off and the gum again treated with diethyl ether. No solid formed. The gum was dried under reduced pressure and the product, D-desthiobiotinamidocaproate N-hydroxy succinimidyl ester was used directly in the next dye coupling reaction, assuming a theoretical yield of 62 mg.

3.5 Preparation of α-D-Desthiobiotin-ε-Cy5-L-lysine-OH [N⁶-(6-(2Z)-2-[(2E,4E)-5-(1-ethyl-3,3-dimethyl-5-sulfo-3H-indolium-2-yl)penta-2,4-dienylidenel-3.3-dimethyl-5-sulfo-2,3-dihydro-1H-indol-1-yl]hexanoyl)-N²-(6-{[6-(5-methyl-2-oxoimidazolidin-4-yl)hexanoyl]amino}hexanoyl)lysine]

ε-Cy5-L-lysine-OH (43 mg, 0.048 mmol), D-desthiobiotinamidocaproate N-hydroxy succinimidyl ester (62 mg, 0.146 mmol) and DIEA (80 μl, 0.45 mmol) were stirred together in DMF (2 ml) for 3 hrs. TLC (RP C18; 1:1 MeOH:water) showed the formation of a new product spot, rf=0.79, just under that of the starting material. The product was precipitated into diethyl ether (200 ml) and then filtered off. The material was purified in multiple runs by HPLC [Vydac Protein and Peptide C18 column; eluent A=0.1% TFA/water, eluent B=0.1% TFA/MeCN, various gradients, detection at 215 nm) until the material was seen to be pure by ¹H NMR. Analytical HPLC AKTA analysis; Phenomenex C18 column; 0-50% B over 30 mins at 1 ml/min; eluent A=0.1% TFA/water, eluent B=0.1% TFA/MeCN, detection at 650 nm; rt=22.04 mins. MS (MALDI TOF) found 1094 (M⁺); [theoretical (C₅₅H₈₀N₇O₁₂S₂) 1094].

3.6 Preparation of α-D-Desthiobiotin-c-Cy5-L-lysine-MESNA [N⁶-(6-{(2Z)-2-[(2E,4E)-5-(1-ethyl-3,3-dimethyl-5-sulfo-3H-indolium-2-yl)penta-2,4-dienylidene]-3,3-dimethyl-5-sulfo-2,3-dihydro-1H-indol-1-yl}hexanoyl)-N²-(6-{[6-(5-methyl-2-oxoimidazolidin-4-yl)hexanoyl]amino}hexanoyl)lysylthio propane-3-sulfonic acid]

α-D-Desthiobiotin-F-Cy5-L-lysine-OH (10 mg, 8.8 μmol) was dissolved in anhydrous dimethylsulphoxide (2 ml), PyAOP (10 mg, 19.2 μmol), MESNA (5 mg, 0.30 mmol) and DIEA (10 μl, 0.06 mmol) were added and the reaction mixture was stirred under nitrogen for 4 hrs. The reaction mixture was purified by RP-HPLC; [Vydac Protein and Peptide C18 column; 15-40% B over 45 mins at 10 ml/min; eluent A=0.1% TFA/water, eluent B=0.1% TFA/MeCN, detection at 215 nm). The product containing fractions were combined and the majority of solvent removed under reduced pressure, the residue was freeze dried. The product was obtained as a fluffy blue solid (4 mg, 37%). TLC; RP C18; 1:1 water:acetonitrile) rf=0.76. Analytical HPLC AKTA analysis; Phenomenex C18 column; 0-50% B over 30 mins at 1 ml/min; eluent A=0.1% TFA/water, eluent B=0.1% TFA/MeCN, detection at 650 nm; rt=21.04 mins. λmax=648 nm (PBS buffer). MS (MALDI TOF) found 1219 (MH⁺); [theoretical (C₅₇H₈₄N₇O₁₄S₄) 1218].

4. Protein Labelling and Affinity Purification

4.1 Labelling of N Terminal Cysteine Grb2SH2 with α-D-desthiobiotin-ε-Cy5-L-lysine-MESNA and Purification

To N-terminal cysteine Grb2SH2 (N-Cys-Grb2SH2) (200 μM in PBS buffer pH 7.2) (200 μl) was added α-D-desthiobiotin-c-Cy5-L-lysine-MESNA (2 mM in reaction buffer) (200 μl). N-Cys-Grb2SH2 was prepared using recombinant techniques. The reaction buffer consisted of phosphate buffer (200 mM), pH 7.2 containing sodium chloride (200 mM) and 4% MESNA. The reaction mixture was left at RT for 12 hrs, wrapped in foil to protect from light. The reaction was then quenched with dl-dithiothreitol (final concentration 60 mM). Unreacted dye was separated from labelled/unlabelled protein by FPLC, using a fast desalt column and eluent of PBS buffer, pH 7.4; 2 mVmin, detection 280 nm. Protein fractions were combined and desthiobiotin-e-Cy5-L-lysine affinity probe labelled protein was bound to streptavidin beads (PIERCE Ultralink™ streptavidin). The beads were washed vigorously with both PBS buffer and binding buffer (PBS containing 500 mM NaCl). The product; α-D-desthiobiotin-ε-Cy5-L-Lys-Cys-Grb2SH2 was extracted from the streptavidin beads by adding cold biotin (1.6 mM). Several extraction runs were required. The materials were further purified by dialysis (PIERCE Slide-a-lyser™ mini dialysis units, 7,000 mwco) to remove free biotin from the sample. The product was analysed by SDS PAGE together with the following controls (see FIG. 1):

| | |
|---|---|
| Lane 1 | MW marker |
| Lane 2 | Unligated protein control |
| Lane 3 | Ligation reaction mixture: α-D- |
| | desthiobiotin-ε-Cy5-L-Lysine- |
| | MESNA and N-Cys-Grb2SH2 |
| Lane 4 | Labelled/unlabelled N-Cys-Grb2SH2 |
| | after FPLC purification |
| Lane 5 | Unbound protein |
| Lane 6 | Streptavidin bead washes |
| Lanes 7, 8, 9 | Affinity purified product |
| Lane 10 | Unreacted α-D-desthiobiotin-ε- |
| | Cy5-L-Lysine-MESNA |

The gel was imaged using a Typhoon imager (FIG. 1B) using parameters for Cy5 fluorescence to detect fractions containing the fluorescent label. The gel was then stained with Coomassie blue stain (FIG. 1A) to determine the protein containing fractions. SDS PAGE gel shows that (a) unlabelled protein (both factor XA and N-Cys-Grb2SH2 did not bind to the streptavidin beads (FIGS. 1A and 1B, column 5) (enriched protein stain) and (b) the product was removed from the streptavidin beads by adding cold biotin (FIGS. 1A and 1B, columns 7 and 8) (both protein stain and Cy5 fluorescence).

1. A compound comprising a cyanine dye or derivative thereof containing at least one target bonding group selected from a carboxylic acid thioester group or a group suitable for covalent reaction with a thioester, wherein said compound includes an affinity tag covalently bound thereto.

2. The compound of claim 1, having the formula (I): 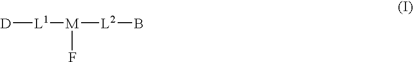
wherein:
D is a dye selected from a cyanine dye or a derivative thereof;
B is an affinity tag;
F comprises a target bonding group selected from a carboxylic acid thioester group and a 1,2-aminothiol group;
M is a group adapted for attaching to F; and
L¹ and L² each independently include a group containing from 1-40 linked atoms selected from carbon atoms which may optionally include one or more groups selected from the group consisting of —NR′—, —O—, —CH═CH—, —CO—NH— and phenylenyl groups, where R′ is selected from hydrogen and C₁-C₄ alkyl.

3. The compound of claim 2, wherein each of L¹ and L² contains from 2 to 30 atoms.

4. The compound of claim 2, wherein L¹ and L² are independently selected from the group consisting of:
—{(CHR′)ₚ-Q-(CHR′)ᵣ}ₛ—
where Q is selected from the group consisting of: —CHR′—, —NR′—, —O—, —CH═CH—, —Ar— and —CO—NH—; R′ is hydrogen or C₁-C₄ alkyl, p is 0-5, r is 1-5 and s is 1 or 2.

5. The compound of claim 4, wherein Q is selected from the group consisting of —CHR′—, —O— and —CO—NH—, where R′ is hereinbefore defined.

6. The compound of claim 1, wherein said affinity tag is selected from the group consisting of biotin and desthiobiotin.

7. The compound of claim 1, wherein said affinity tag is selected from the group consisting of his-tag, iminodiacetic acid and nitrilotriacetic acid.

8. The compound of claim 2, wherein the target bonding group F is a carboxylic acid thioester of formula: 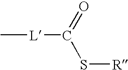
wherein L′ is a bond or is a group containing from 1-30 linked atoms selected from the group consisting of carbon atoms and carbon atoms including one or more groups selected from the group consisting of —NH—, —O— and —CO—NH—; and R″ is C₁-C₄ alkyl, C₆-C₁₀ aryl, or C₇-C₁₅ aralkyl, which may be optionally substituted with sulphonate; or is the group —(CH₂)₂—CONH₂.

9. The compound of claim 2, wherein the target bonding group F is a 1,2-aminothiol group of formula: 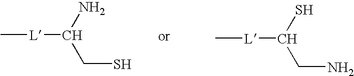
wherein L′ is a bond or is a group containing from 1-30 linked atoms selected from the group consisting of carbon atoms and carbon atoms including one or more groups selected from the group consisting of —NH—, —O— and —CO—NH—.

10. The compound of claim 2, having the formula (II): 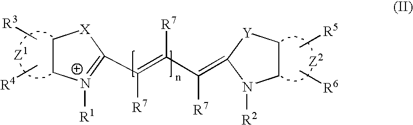
wherein:
groups R³ and R⁴ are attached to the Z¹ ring structure and groups R⁵ and R⁶ are attached to the Z² ring structure;
n is an integer from 1 to 3;
Z¹ and Z² independently represent the atoms necessary to complete one ring or two fused ring aromatic or heteroaromatic systems, each ring having five or six atoms selected from carbon atoms and optionally no more than two atoms selected from oxygen, nitrogen and sulphur;
X and Y are the same or different and are selected from: >CR⁸R⁹, oxygen, sulphur, —CH═CH—, >N—W wherein N is nitrogen and W is selected from hydrogen and the group R¹⁰;
at least one of groups R¹, R², R³, R⁴, R⁵, R⁶, R⁸, R⁹ and R¹⁰ is the group: 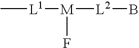
groups R⁷ are independently selected from the group consisting of hydrogen and C₁-C₄ alkyl which may be unsubstituted or substituted with aryl, or two or more of R⁷ together with the group: 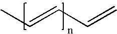
form a hydrocarbon ring system substituted with R⁷ and which may optionally contain a heteroatom selected from —O—, —S— or >NR⁷;
remaining groups R³, R⁴, R⁵ and R⁶ are independently selected from the group consisting of hydrogen, halogen, amide, cyano, nitro, mono- or di-C₁-C₆ alkyl-substituted amino, carbonyl, carboxyl, C₁-C₆ alkyl, C₁-C₆ alkoxy, aryl, heteroaryl, aralkyl and the group —(CH₂)ₘ—Y where Y is selected from sulphonate, sulphate, phosphonate, phosphate and quaternary ammonium and m is zero or an integer from 1 to 6;
remaining groups R⁸, R⁹ and R¹⁰ are independently C₁-C₆ alkyl; and
remaining groups R¹ and R² are independently selected from hydrogen, C₁-C₁₀ alkyl, the group —(CH₂)ₘ—Y wherein Y and m are hereinbefore defined, and benzyl which may be unsubstituted or substituted by up to two nitro groups.

11. The compound of claim 2 having the formula (III): 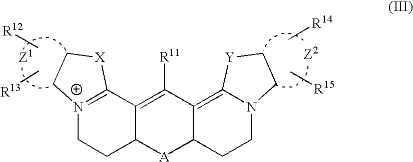
wherein
groups R¹², R¹³, R¹⁴ and R¹⁵ are attached to the rings containing X and Y or, optionally are attached to atoms of the Z¹ and Z² ring structures;
Z¹ and Z² independently represent the atoms necessary to complete one ring or two fused ring aromatic or heteroaromatic systems, each ring having five or six atoms selected from carbon atoms and optionally no more than two atoms selected from oxygen, nitrogen and sulphur;
X and Y are the same or different and are selected from: >CR⁸R⁹, oxygen, sulphur, —CH═CH—, >N—W wherein N is nitrogen and W is selected from hydrogen and the group R¹⁰;
A is selected from O and NR¹⁶ where R¹⁶ is the substituted amino radical: 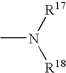
at least one of groups R⁸, R⁹, R¹⁰, R¹¹, R¹², R¹³, R¹⁴, R¹⁵, R¹⁷ and R¹⁸ is the group: 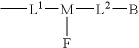
remaining groups R¹¹, R¹², R¹³, R¹⁴ and R¹⁵ are independently selected from the group consisting of hydrogen, halogen, amide, cyano, nitro, mono- or di-C₁-C₆ alkyl-substituted amino, carbonyl, carboxyl, C₁-C₆ alkyl, C₁-C₆ alkoxy, aryl, heteroaryl, aralkyl and the group —(CH₂)ₘ—Y where Y is selected from sulphonate, sulphate, phosphonate, phosphate and quaternary ammonium and m is zero or an integer from 1 to 6;
remaining groups R⁸, R⁹ and R¹⁰ are independently C₁-C₆ alkyl;
remaining group R¹⁷ is selected from hydrogen, C₁-C₄ alkyl and aryl; and remaining group R¹⁸ is selected from C₁-C₆ alkyl, aryl, heteroaryl, an acyl radical having from 2-7 carbon atoms, and a thiocarbamoyl radical.

12. The compound of claim 10, wherein Z¹ and Z² are selected independently from the group consisting of phenyl, pyridinyl, naphthyl, quinolinyl and indolyl moieties.

13. The compound of claim 110 wherein Z¹ and Z² are selected from phenyl and naphthyl moieties.

14. A method for labelling a protein of interest wherein said protein contains or is derivatised to contain an N-terminal cysteine, the method comprising:
i) adding to a liquid containing said protein a compound of formula (I): 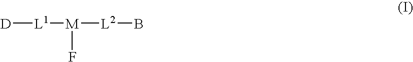
wherein:
D is a dye selected from a cyanine dye or a derivative thereof;
B is a bioaffinity tag;
F comprises a target bonding group selected from a carboxylic acid thioester group and a 1,2-aminothiol group;
M is a group adapted for attaching to F; and
L¹ and L² each independently include a group containing from 1-40 linked atoms selected from carbon atoms which may optionally include one or more groups selected from —NR′—, —O—, —CH═CH—, —CO—NH— and phenylenyl groups, where R′ is selected from hydrogen and C₁-C₄ alkyl; and
ii) incubating said compound with said protein under conditions suitable for labelling said protein.

15. The method of claim 14, wherein each of L¹ and L² contains from 2 to 30 atoms.

16. The method of claim 14, wherein L¹ and L² are independently selected from the group:
—{(CHR′)ₚ-Q-(CHR′)ᵣ}ₛ—
where Q is selected from the group consisting of: —CHR′—, —NR′—, —O—, —CH═CH—, —Ar— and —CO—NH—; R′ is hydrogen or C₁-C₄ alkyl, p is 0-5, r is 1-5 and s is 1 or 2.

17. The method of claim 16, wherein Q is selected from the group consisting of —CHR′—, —O— and —CO—NH—, where R′ is hereinbefore defined.

18. The method of claim 14, further comprising separating and/or purifying the dye-labelled protein of interest by affinity chromatography.

19. The method of claim 14, wherein said protein of interest is selected from antibody, the group consisting of antibodies, antigens, proteins peptides, microbial materials, cells and cell membranes.

20. The compound of claim 11, wherein Z¹ and Z² are selected independently from the group consisting of phenyl, pyridinyl, naphthyl, quinolinyl and indolyl moieties.

21. The compound of claim 11, wherein Z¹ and Z² are selected from phenyl and naphthyl moieties.
